(19) United States
(12) Patent Application Publication
(10) Pub. No.: US 2022/0039575 A1
(43) Pub. Date: Feb. 10, 2022

(54) MULTIFUNCTIONAL KNIFE HOLDER

(71) Applicant: Wai Chi Raymond Yu, Hong Kong (HK)

(72) Inventor: Wai Chi Raymond Yu, Hong Kong (HK)

(21) Appl. No.: 17/150,165

(22) Filed: Jan. 15, 2021

(30) Foreign Application Priority Data
Aug. 4, 2020 (CN) 202010770759.3

(51) Int. Cl. A47G 21/14

(52) U.S. Cl. CPC A47G 21/14; A47G 2200/106

(57) ABSTRACT

A multifunctional knife holder is disclosed, which includes a falling prevention base, a knife holder body, a top cover and a locking bar. The knife holder body is of a cylinder structure with an opening at a top thereof and with a cavity inside. A plurality of partition plates are provided inside the knife holder body, are vertically arranged at intervals and divide the cavity into a plurality of sub-chambers. The top cover is connected with the top of the knife holder body and provided with a plurality of through slots communicated with the sub-chambers. A tension spring is provided on an inner wall of the knife holder body. The locking bar is connected with a plurality of magnetic pressing plates arranged at intervals along a length direction of the locking bar. The locking bar is disposed through the plurality of partition plates and abuts against the tension spring.

### CROSS-REFERENCE TO RELATED APPLICATIONS

This application claims priority to Chinese Patent Application No. 202010770759.3 with a filing date of Aug. 4, 2020. The content of the aforementioned applications, including any intervening amendments thereto, are incorporated herein by reference.

### TECHNICLAL FIELD

The disclosure relates to the technical field of a kitchen appliance, in particular to a multifunctional knife holder.

### BACKGROUND ART

A knife is one of commonly used appliances in kitchen, which is usually inserted into a knife holder for storage. However, for many knife holders, there is no fixed connection for the knife holder itself and there is no fixed connection between the knife and the knife holder, once the knife holder is knocked down by a playing child or accidentally in daily operations, the knife is very easy to slip off the knife holder and hurt people.

### SUMMARY

The disclosure provides a multifunctional knife holder, which can solve a problem that a knife is easy to slide off the knife holder.

An embodiment of the disclosure is realized as follows:

The disclosure provides a multifunctional knife holder, which includes a falling prevention base, a knife holder body, a top cover and a locking bar.

The knife holder body is of a cylinder structure with an opening at a top thereof and with a cavity inside. A plurality of partition plates are provided inside the knife holder body, and the plurality of partition plates are vertically arranged at intervals and divide the cavity into a plurality of sub-chambers.

The top cover is connected with the top of the knife holder body, and is provided with a plurality of through slots communicated with the sub-chambers. Each through slot corresponds to one sub-chamber.

A tension spring is provided on an inner wall of the knife holder body. The locking bar is connected with a plurality of magnetic pressing plates which are arranged at intervals along a length direction of the locking bar. An end of the locking bar is arranged outside the knife holder body, and the locking bar is disposed through the plurality of partition plates and abuts against the tension spring. When the locking bar abuts against the tension spring, each of the magnetic pressing plates is located in one of the sub-chambers and corresponds to the through slot, so that when the knife is inserted into the sub-chamber from the through slot, a side surface of the knife abuts against the magnetic pressing plate.

Furthermore, an end of the through slot is provided with a grind stone, and the grind stone is provided with a wedge-shaped notch which can match with a cutting edge of the knife.

Furthermore, a sealing strip is provided on a side wall of the through slot, and an end of the sealing strip is connected with the grind stone.

Furthermore, a side surface of the top cover is provided with a vent hole.

Furthermore, an end of each sub-chamber proximate to the opening is provided with at least one pair of cleaning rollers, and central axes of the cleaning rollers are parallel with the partition plate.

Furthermore, the cleaning roller is mounted on the knife holder body with an elastic connection box. The elastic connection box includes a box body and a central rotating shaft mounted on a side wall of the knife holder body. A clockwork spring is provided in an inner cavity of the box body, an outer end of the clockwork spring is connected with the box body, and an inner end of the clockwork spring is fixedly connected with the central rotating shaft.

Furthermore, a water filtering mesh plate is provided at a bottom of the inner cavity of the knife holder body. The water filtering mesh plate is arranged to be separated from a bottom wall of the knife holder body.

Furthermore, the water filtering mesh plate is horizontally arranged above the bottom wall of the knife holder body, a drainage hole is provided on the bottom of the side wall of the knife holder body, and the bottom wall of the knife holder body is inclined downward toward the drainage hole.

Furthermore, the falling prevention base includes a connecting pipe, a hose and a suction pad connected with the knife holder body. The suction pad is configured to be adsorbed on the work table. An upper end of the suction pad is fixedly connected with the knife holder body, and the connecting pipe pierces into the inner cavity of the knife holder body from an inner cavity of the suction pad so that the inner cavity of the suction pad is communicated with the inner cavity of the knife holder body, and an end of the connecting pipe located in the inner cavity of the suction pad is connected with the hose. When the suction pad is adsorbed on the work table, the connecting pipe can abut the hose against the work table to close an inner cavity of the hose.

Furthermore, a sterilizing lamp and/or an electric heater are mounted on both the inner wall of the knife holder body and the partition plate.

The multifunctional knife holder has advantages as follows

The knife holder body includes several sub-chambers , which can store many knives; and when the end of the locking bar abuts against the tension spring and the knife is inserted into the sub-chamber from the through slot, the side surface of the knife abuts against the magnetic pressing plate, so that the knife is magnetically attached to the magnetic pressing plate, and even if the knife holder accidentally falls down, the knife is not easy to slip off the knife holder, thus improving operating safety.

### BRIEF DESCRIPTION OF THE DRAWINGS

In order to illustrate technical schemes of embodiments of this disclosure more clearly, the following drawings used in the embodiments will be briefly introduced; it should be understood that the following drawings only show some embodiments of this disclosure, therefore they should not be regarded as a limiting of the protection scope; and other related drawings can be obtained according to these ones without creative effort for ordinary skilled in the art.

FIG. 1 is a schematic structural diagram of a multifunctional knife holder according to an embodiment of the present disclosure;

FIG. 2 is a structural schematic diagram of a knife inserted into a multifunctional knife holder according to an embodiment of the present disclosure;

FIG. 3 is a schematic structural diagram of a top cover according to an embodiment of the present disclosure;

FIG. 4 is a structural schematic diagram of a cleaning roller according to an embodiment of the present disclosure;

FIG. 5 is a schematic structural diagram of an elastic connection box according to an embodiment of the present disclosure; and

FIG. 6 is a schematic diagram of the internal structure of the knife holder body according to an embodiment of the present disclosure.

Reference Number: 1—Falling Prevention Base; 10—Suction Pad; 11—Connecting Pipe; 12—Hose; 2—Knife Holder Body; 20—Drainage Hole; 21—Partition Plate; 211—Sub-chamber; 22—Cleaning Roller; 221—Elastic Connection Box; 2211—Box Body; 2212—Clockwork Spring; 222—Roller; 223—Cleaning Cover; 224—Central Rotating Shaft; 23—Sterilizing Lamp; 24—Electric Heater; 25—Water Filtering Mesh Plate; 26—Tension Spring; 27—Magnetic Pressing Plate; 28—Locking Bar; 29—Handle; 3—Top Cover; 31—Vent Hole; 32—Grind Stone; 321—Wedge-shaped Notch; 33—Sealing Strip; 34—Through Slot; 4—Knife.

### DETAILED DESCRIPTION

In order to make the purposes, technical schemes and advantages of embodiments of this disclosure more clear, the technical schemes in the embodiments of this disclosure will be described clearly and completely with reference to the drawings in the embodiments of this disclosure; and it is obvious that the described embodiments are part of the embodiments of this disclosure, but not all of them. Generally, components of the embodiments of the present disclosure described and illustrated in the drawings herein may be arranged and designed in various different configurations.

Therefore, the following detailed description of the embodiments of the disclosure provided in the drawings is not intended to limit the claimed scope of the application, but only shows selected embodiments of the disclosure. On a basis of the embodiments in this disclosure, all other embodiments obtained by the ordinary skilled in the art without any creative effort are within the protection scope of this disclosure.

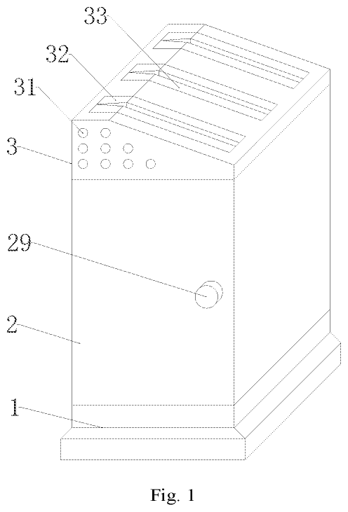

The embodiment of the disclosure provides a multifunctional knife holder. Referring to FIG. 1, the knife holder includes a falling prevention base 1, a knife holder body 2, a top cover 3 and a locking bar 28.

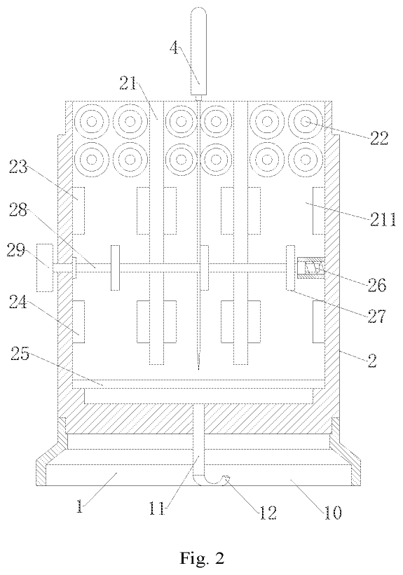

Referring to FIGS. 1 and 2, the knife holder body 2 is of a cylinder structure with an opening at a top thereof and with a cavity inside. A plurality of partition plates 21 are provided inside the knife holder body 2, and the plurality of partition plates 21 are vertically arranged at intervals and divide the cavity into a plurality of sub-chambers 211.

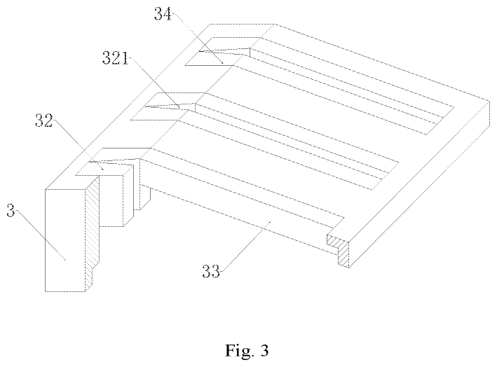

Referring to FIGS. 1 and 3, the top cover 3 is connected with the top of the knife holder body 2, and is provided with a plurality of through slots 34 communicated with the sub-chambers 211. Each through slot 34 corresponds to one sub-chamber 211. That is to say, when the knife 4 is inserted into the through slot 34, it can enter one of the sub-chambers 211. Since there are a plurality of sub-chambers 211 in the knife holder body 2, a plurality of knifes 4 can be stored. Optionally, the through slot 34 is a rectangular through slot 34.

Referring to FIG. 2, a tension spring 26 is provided on an inner wall of the knife holder body 2. The locking bar 28 is connected with a plurality of magnetic pressing plates 27 which are arranged at intervals along a length direction of the locking bar 28. An end of the locking bar 28 is arranged outside the knife holder body 2, and the locking bar 28 is disposed through the plurality of partition plates 21 and abuts against the tension spring 26. When the locking bar 28 abuts against the tension spring 26, each of the magnetic pressing plates 27 is located in one of the sub-chambers 211 and corresponds to the through slot 34, so that when the knife 4 is inserted into the sub-chamber 211 from the through slot 34, a side surface of the knife 4 abuts against the magnetic pressing plate 27. Optionally, an end of the locking bar 28 away from the tension spring 26 is connected with a handle 29, and the handle 29 is arranged outside the knife holder body 2. The handle 29 can be operated to slide the locking bar 28 so that the other end of the locking bar 28 abuts against the tension spring 26, which is more convenient to use.

When the other end of the locking bar 28 abuts against the tension spring 26 and the knife 4 is inserted into the sub-chamber 211 from the through slot 34, the side surface of the knife 4 abuts against the magnetic pressing plate 27, so that the knife 4 is magnetically attached to the magnetic pressing plate 27, and even if the knife holder accidentally falls down, the knife 4 is not easy to slip off the knife holder, thus improving operation safety.

Referring to FIG. 3, in an embodiment, an end of the through slot 34 is provided with a grind stone 32, and the grind stone 32 is provided with a wedge-shaped notch 321 which can match with a cutting edge of the knife 4. When the knife 4 is inserted into the through slot 34, the cutting edge of the knife 4 is ground against the grindstone 32 through the wedge-shaped notch 321, so that the cutting edge of the knife 4 can be sharpened.

Optionally, a sealing strip 33 is provided on a side wall of the through slot 34, and an end of the sealing strip 33 is connected with the grind stone 32. When the knife 4 is inserted into the through slot 34, the side surface of the knife 4 can contact with the sealing strip 33, thereby better preventing the knife 4 from slipping off the knife holder. In addition, in order to keep the knife holder body 2 dry, a side surface of the top cover 3 is provided with a vent hole 31, which facilitates humid gas to be exhausted from the vent hole.

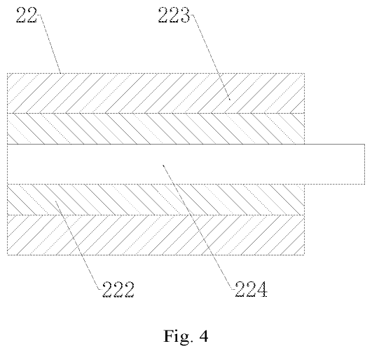

Further, Referring to FIG. 4, in an embodiment, an end of each sub-chamber 211 proximate to the opening is provided with at least one pair of cleaning rollers 22, and central axes of the cleaning rollers 22 are parallel with the partition plate 21. In addition, it should be noted that a pair of cleaning rollers 22 means two cleaning rollers 22 arranged in parallel. Optionally, at least two pairs of cleaning rollers 22 are correspondingly arranged for each through slot 34. Exemplarily, the cleaning roller 22 includes a central rotating shaft 224 mounted on the side wall of the knife holder body 2, the central rotating shaft 224 is provided with a roller 222, and a cleaning cover 223 is arranged outside of the roller 222. A pair of cleaning rollers 22 are arranged corresponding to one through slot 34, so that when the knife 4 is inserted into the through slot 34, the pair of cleaning rollers 22 can abrade and clean the knife 4 and wipe water spots on surfaces of the knife 4.

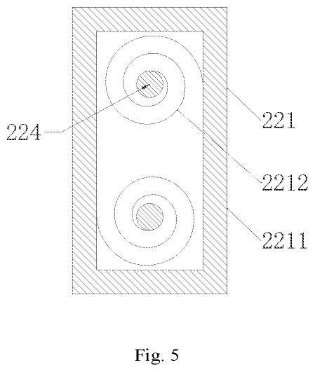

Referring to FIG. 5, optionally, the cleaning roller 22 is mounted on the knife holder body 2 with an elastic connection box 221. The elastic connection box 221 includes a box body 2211. A clockwork spring 2212 is provided in an inner cavity of the box body 2211, an outer end of the clockwork spring 2212 is connected with the box body 2211, and an inner end of the clockwork spring 2212 is fixedly connected with the central rotating shaft 224.

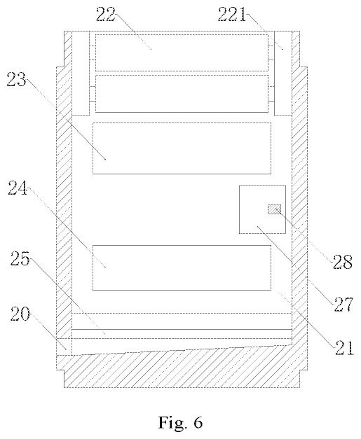

Further, referring to FIG. 6, in an embodiment, a water filtering mesh plate 25 is provided at a bottom of the inner cavity of the knife holder body 2. The water filtering mesh plate 25 is arranged to be separated from a bottom wall of the knife holder body 2.

Optionally, the water filtering mesh plate 25 is horizontally arranged above the bottom wall of the knife holder body 2, a drainage hole 20 is provided on the bottom of the side wall of the knife holder body 2, and the bottom wall of the knife holder body 2 is inclined downward toward the drainage hole 20. When the knife 4 is inserted into the knife holder body 2, water remained on the knife 4 flows to the bottom wall of the knife holder body 2 through the water filtering mesh plate 25. Since the bottom wall of the knife holder body 2 is inclined downward toward the drainage hole 20, the water can flow from the bottom wall of the knife holder body 2 to the drainage hole 20 so as to be discharged through the drainage hole 20.

Optionally, a sterilizing lamp 23 and/or an electric heater 24 are mounted on both the inner wall of the knife holder body and the partition plate 21. The sterilizing lamp 23 can sterilize, and the electric heater 24 can heat to evaporate moisture and dry the knife 4, thus keeping the knife 4 cleaner.

Furthermore, still referring to FIG. 2, in an embodiment, the falling prevention base 1 includes a connecting pipe 11, a hose 12 and a suction pad 10 connected with the knife holder body 2. The suction pad 10 is configured to be adsorbed on the work table. An upper end of the suction pad 10 is fixedly connected with the knife holder body 2, and the connecting pipe 11 pierces into the inner cavity of the knife holder body 2 from an inner cavity of the suction pad 10 so that the inner cavity of the suction pad 10 is communicated with the inner cavity of the knife holder body 2, and an end of the connecting pipe 11 located in the inner cavity of the suction pad 10 is connected with the hose 12. When the suction pad 10 is adsorbed on the work table, the connecting pipe 11 can abut the hose 12 against the work table to close an inner cavity of the hose 12.

When the suction pad 10 is adsorbed on the work table, the connecting pipe 11 abuts the hose 12 against the work table to close the inner cavity of the hose 12, so that the falling prevention base 1 is firmly adsorbed on the work table and is not easy to fall down.

To sum up, the multifunctional knife holder according to the embodiment of the present disclosure is not easy to fall down, and additionally, even if the knife holder accidentally falls down, the side surface of the knife 4 abuts against the magnetic pressing plate 27, so that the knife 4 is magnetically attracted to the magnetic pressing plate 27, and it is not easy for the knife 4 to slide off the knife holder, thus improving the operation safety. Furthermore, the cleaning roller 22 can abrade and clean the knife 4 and wipe the water spots on the surfaces of the knife 4. The electric heater 24 can dry the knife 4, and the sterilizing lamp 23 can sterilize, thus keeping the knife 4 cleaner. In addition, the water on the knife 4 can also flow from the bottom wall of the knife holder body 2 to the drainage hole 20 through the water filtering mesh plate 25 so as to be discharged through the drainage hole 20.

The above is only specific embodiments of the present disclosure, and is not intended to limit this disclosure, and modifications and variations can be made in this disclosure for those skilled in the art. Any modification, equivalent substitution, improvement, etc. made within the spirit and principle of this disclosure shall be encompassed within the protection scope of this disclosure.

I claim:

1. A multifunctional knife holder, comprising a falling prevention base, a knife holder body, a top cover and a locking bar; wherein
the knife holder body is of a cylinder structure with an opening at a top thereof and with a cavity inside, a plurality of partition plates are provided inside the knife holder body, and the plurality of partition plates are vertically arranged at intervals and divide the cavity into a plurality of sub-chambers;
the top cover is connected with the top of the knife holder body, and is provided with a plurality of through slots communicated with the sub-chambers, each through slot corresponding to one sub-chamber; and
a tension spring is provided on an inner wall of the knife holder body, the locking bar is connected with a plurality of magnetic pressing plates which are arranged at intervals along a length direction of the locking bar, an end of the locking bar is arranged outside the knife holder body, and the locking bar is movably disposed through the plurality of partition plates and abuts against the tension spring, when the locking bar abuts against the tension spring, each of the magnetic pressing plates is located in one of the sub-chambers and corresponds to the through slot, so that when the knife is inserted into the sub-chamber from the through slot, a side surface of the knife abuts against the magnetic pressing plate.

2. The multifunctional knife holder according to claim 1, wherein an end of the through slot is provided with a grind stone, and the grind stone is provided with a wedge-shaped notch which matches with a cutting edge of the knife.

3. The multifunctional knife holder according to claim 2, wherein a sealing strip is provided on a side wall of the through slot, and an end of the sealing strip is connected with the grind stone.

4. The multifunctional knife holder according to claim 1, wherein a side surface of the top cover is provided with a vent hole.

5. The multifunctional knife holder according to claim 1, wherein an end of each sub-chamber proximate to the opening is provided with at least one pair of cleaning rollers, and central axes of the cleaning rollers are parallel with the partition plate.

6. The multifunctional knife holder according to claim 5, wherein the cleaning roller is mounted on the knife holder body with an elastic connection box, the elastic connection box comprises a box body and a central rotating shaft mounted on a side wall of the knife holder body, a clockwork spring is provided in an inner cavity of the box body, an outer end of the clockwork spring is connected with the box body, and an inner end of the clockwork spring is fixedly connected with the central rotating shaft.

7. The multifunctional knife holder according to claim 1, wherein a water filtering mesh plate is provided at a bottom of an inner cavity of the knife holder body, and the water filtering mesh plate is arranged to be separated from a bottom wall of the knife holder body.

8. The multifunctional knife holder according to claim 7, wherein the water filtering mesh plate is horizontally arranged above the bottom wall of the knife holder body, a drainage hole is provided on the bottom of the side wall of the knife holder body, and the bottom wall of the knife holder body is inclined downward toward the drainage hole.

9. The multifunctional knife holder according to claim 1, wherein the falling prevention base includes a connecting pipe, a hose, and a suction pad connected with the knife holder body, wherein the suction pad is configured to be adsorbed on a work table, an upper end of the suction pad is fixedly connected with the knife holder body, the connecting pipe pierces into an inner cavity of the knife holder body from an inner cavity of the suction pad so that the inner cavity of the suction pad is communicated with the inner cavity of the knife holder body, an end of the connecting pipe located in the inner cavity of the suction pad is connected with the hose, when the suction pad is adsorbed on the work table, the connecting pipe abuts the hose against the work table to close an inner cavity of the hose.

10. The multifunctional knife holder according to claim 1, wherein a sterilizing lamp is mounted on both an inner wall of the knife holder body and the partition plate.

11. The multifunctional knife holder according to claim 1, wherein an electric heater is mounted on both an inner wall of the knife holder body and the partition plate.
